(19) United States
(12) Patent Application Publication
(10) Pub. No.: US 2020/0387590 A1
(43) Pub. Date: Dec. 10, 2020

(54) ENDOSCOPE SYSTEM, PROCESSOR, CONTROL METHOD, AND COMPUTER-READABLE RECORDING MEDIUM

(71) Applicant: OLYMPUS CORPORATION, Tokyo (JP)

(72) Inventors: Yugo KOIZUMI, Yokohama-shi (JP); Hidekazu SHINANO, Tokyo (JP); Hideyuki KUGIMIYA, Tokyo (JP)

(73) Assignee: OLYMPUS CORPORATION, Tokyo (JP)

(21) Appl. No.: 16/883,111

(22) Filed: May 26, 2020

Related U.S. Application Data
(63) Continuation of application No. PCT/JP2018/034033, filed on Sep. 13, 2018.

(30) Foreign Application Priority Data
Nov. 27, 2017 (JP) 2017-227191

(51) Int. Cl. G06F 21/32; G06F 3/16; G06F 21/44; H04W 12/08; H04W 12/06; G16H 30/40; G16H 40/40; G16H 40/67; G05B 19/4155; A61B 1/00

(52) U.S. Cl. CPC G06F 21/32; G06F 3/16; G06F 21/44; H04W 12/08; H04W 12/06; G16H 30/40; G06K 9/00013; G16H 40/67; G05B 19/4155; A61B 1/00009; A61B 1/00006; A61B 1/00016; A61B 1/00059; G16H 40/40

(57) ABSTRACT

An endoscope system includes: a processor that is connectable to a peripheral device; and a terminal device configured to communicate with the processor. The terminal device is configured to transmit terminal identification information, and an authentication result indicating whether a user of the terminal device is a registered user that has been pre-registered. The processor includes a communication circuit configured to transmit processor identification information, and authentication information that enables mutual communication, to the terminal device, and receive the terminal identification information and the authentication result from the terminal device; a connection determining circuit configured to determine whether the terminal device is a destination that is enabled to perform mutual communication based on the authentication result; and a communication control circuit configured to enable communication between the terminal device and the peripheral device based on a determination result of the connection determining circuit and on the authentication result.

### CROSS-REFERENCE TO RELATED APPLICATION

This application is a continuation of PCT international application Ser. No. PCT/JP2018/034033, filed on Sep. 13, 2018 which designates the United States, incorporated herein by reference, and which claims the benefit of priority from Japanese Patent Applications No. 2017-227191, filed on Nov. 27, 2017, incorporated herein by reference.

### BACKGROUND

The present disclosure relates to an endoscope system, a processor, a control method, and a computer-readable recording medium to display image data obtained by capturing an inside of a body of a subject by inserting an endoscope into the subject.

In recent years, a technique for supporting examination-related operations and cleaning-related operations by respectively setting an examination schedule and a cleaning schedule of an endoscope, and by notifying as scheduled to a medical staff including a doctor in a system for supporting an endoscopic examination operations has been known (for example, JP-A-2017-117295). This technique helps keeping on a schedule of endoscopy operations appropriately by informing about actions to be performed at determined informing timing based on time specified by scheduled examination-start-time information included in an examination schedule, and on timing information to inform about actions to be performed by a medical staff before start of the examination, and by thereafter receiving a confirmation from the medical staff.

### SUMMARY

An endoscope system according to the disclosure includes: a processor configured to perform image processing on endoscopic image data acquired by an endoscope that observes inside a subject, the processor being connectable to a peripheral device; and a terminal device configured to communicate with the processor. The terminal device is configured to transmit terminal identification information identifying the terminal device, and an authentication result indicating whether a user of the terminal device is a registered user that has been pre-registered, the processor includes a communication circuit configured to transmit processor identification information identifying the processor, and authentication information that enables mutual communication, to the terminal device, and receive the terminal identification information and the authentication result from the terminal device; a connection determining circuit configured to determine whether the terminal device is a destination that is enabled to perform mutual communication based on the authentication result received by the communication circuit; and a communication control circuit configured to enable communication between the terminal device and the peripheral device based on a determination result of the connection determining circuit and on the authentication result.

A processor according to the disclosure is a processor that performs image processing on endoscopic image data acquired by an endoscope that observes inside a subject, the processor being connectable to a peripheral device. The processor includes: a communication circuit configured to transmit processor identification information identifying the processor, and authentication information that enables mutual communication, to the terminal device configured to communicate with the processor, and receive terminal identification information identifying the terminal, and an authentication result indicating whether a user of the terminal device is a registered user that has been pre-registered, from the terminal device; a connection determining circuit configured to determine whether the terminal device is a destination enabled to perform mutual communication based on the authentication result received by the communication circuit; and a communication control circuit configured to enables communication between the terminal device and the peripheral device based on a determination result of the connection determining circuit and on the authentication result.

A control method according to the disclosure is a control method that is performed by an endoscope system including a processor that performs image processing on endoscopic image data acquired by an endoscope that observes inside a subject, the processor being connectable to a peripheral device, and a terminal device configured to communicate with the processor. The method includes: transmitting processor identification information identifying the processor, and authentication information that enables mutual communication, to the terminal device; receiving terminal identification information identifying the terminal device, and an authentication result indicating whether a user of the terminal device is a registered user that has been pre-registered, from the terminal device; determining whether the terminal device is a destination that is enabled to perform mutual communication based on the authentication result; and enabling communication between the terminal device and the peripheral device based on the determination result and on the authentication result.

A computer-readable recording medium according to the disclosure is a non-transitory computer-readable recording medium with an executable program stored thereon. Provided is the program instructing an endoscope system including a processor that performs image processing on endoscopic image data acquired by an endoscope that observes inside a subject, the processor being connectable to a peripheral device, and a terminal device configured to communicate with the processor to perform: transmitting processor identification information identifying the processor, and authentication information that enables mutual communication, to the terminal device; receiving terminal identification information identifying the terminal device, and an authentication result indicating whether a user of the terminal device is a registered user that has been pre-registered, from the terminal device; determining whether the terminal device is a destination that is enabled to perform mutual communication based on the authentication result; and enabling communication between the terminal device and the peripheral device based on the determination result and on the authentication result.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a block diagram illustrating a functional configuration of an endoscope system according to one embodiment;

FIG. 2 is a block diagram illustrating a functional configuration of a terminal device according to one embodiment;

FIG. 3 is a flowchart showing an overview of processing performed by a processor according to one embodiment;

FIG. 4 is a flowchart showing details of connection processing in FIG. 3; and

FIG. 5 is a flowchart showing details of communication driving processing in FIG. 3.

### DETAILED DESCRIPTION

Hereinafter, embodiments of an endoscope system including an endoscope that captures an inside of a body cavity of a subject, such as a patient, and displays an image will be described. The embodiments are not intended to limit this disclosure. Furthermore, like reference symbols are assigned to like parts throughout the drawings.

Configuration of Endoscope System

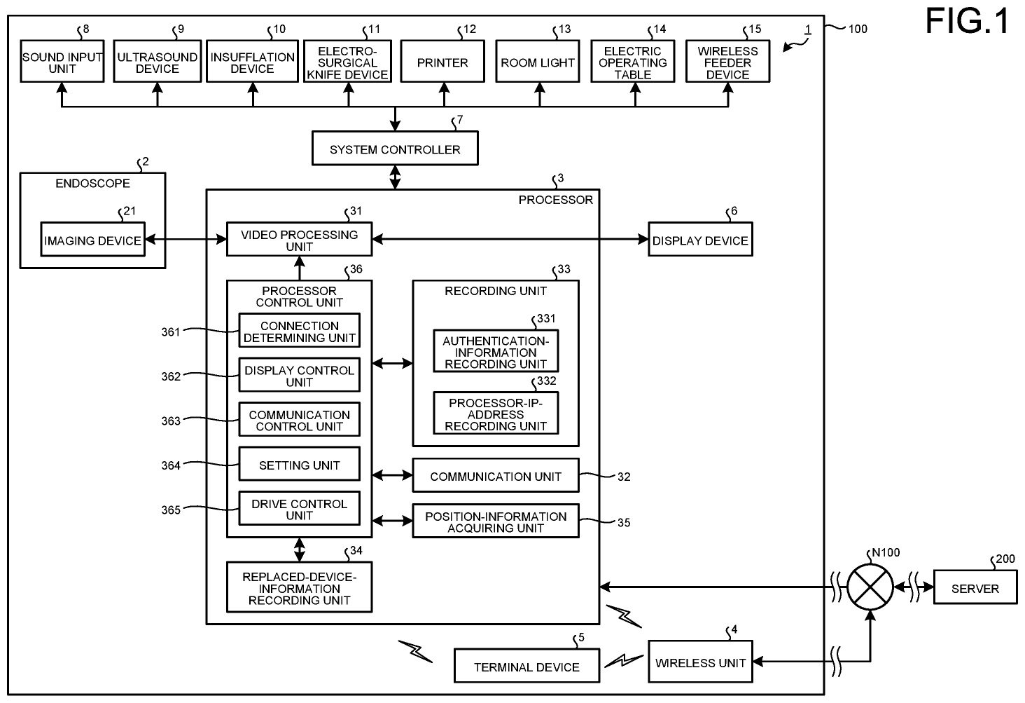

FIG. 1 is a block diagram illustrating a functional configuration of an endoscope system according to one embodiment. An endoscope system 1 illustrated in FIG. 1 is used in an operating room 100 in a hospital when medical staffs including at least a doctor performs an endoscopic surgery, an endoscopic examination, or an endoscopic treatment with respect to a subject, such as a patient. The endoscope system 1 includes an endoscope 2, a processor 3, a wireless unit 4, a terminal device 5, a display device 6, a system controller 7, a sound input unit 8, an ultrasound device 9, an insufflation device 10, an electrosurgical knife device 11, a printer 12, a room light 13, an electric operating table 14, and a wireless feeder device 15.

First, a configuration of the endoscope 2 will be explained. The endoscope 2 is inserted into a body of a subject. The endoscope is constituted of a rigid endoscope or a flexible endoscope. The endoscope 2 illuminates illumination light to an inside of the subject, captures an area inside the subject illuminated with the illumination light to generate endoscopic image data, and outputs this generated endoscopic image data to the processor 3, under control of the processor 3. The endoscope 2 includes an imaging device 21 that generates image data by imaging an inside of the subject. The imaging device 21 is constituted of an image sensor, such as a charge coupled device (CCD) and a complementary metal oxide semiconductor (CMOS), an analog/digital (A/D) converter circuit, or the like. The endoscope 2 is connected to the processor 3 by wired or wireless connection such that mutual communication is possible. Moreover, when the endoscopic image data generated by the endoscope 2 is transmitted wirelessly, the endoscopic image data may be sequentially transmitted to the processor through a wireless unit 4 described later, or may be sequentially transmitted to a server 200 arranged outside the operating room 100 in a hospital through a network N100.

Next, a configuration of the processor 3 will be explained. The processor 3 controls the endoscope 2, and subjects the endoscopic image data sequentially input from the endoscope 2 to predetermined image processing, to sequentially output to the display device 6. The processor 3 includes a video processing unit 31, a communication unit 32, a recording unit 33, a replaced-device-information recording unit 34, a position-information acquiring unit 35, and a processor control unit 36.

The video processing unit 31 subjects the endoscopic image data input from the endoscope 2 to predetermined image processing, to output to the display device 6. The predetermined image processing is synchronization processing, demosaicing processing (when the imaging device 21 has the Bayer arrangement), white balance adjustment processing, γ correction processing, saturation adjustment processing, format conversion processing, and the like. The video processing unit 31 is constituted of a field programmable gate array (FPGA), an application specific integrated circuit (ASIC), a graphics processing unit (GPU), or the like.

The communication unit 32 is constituted of a communication module, and performs mutual communication with the terminal device 5 in accordance with predetermined communication standard. Moreover, the communication unit 32 performs mutual communication with the terminal device 5 through the wireless unit 4, or with the server 200 arranged in the hospital through the network N100. The predetermined communication standard includes Wi-Fi (wireless fidelity) (registered trademark) communication, Bluetooth (registered trademark) communication, and Bluetooth Low Energy (registered trademark) communication (hereinafter, simply “BLE communication”). For example, in the case of Wi-Fi, assuming a local network, there is a relationship of an access point and a station as roles of devices, and there is a relationship that a station is connected to a wireless network established by the access points as overall connection processing. As a general connection sequence, the wireless unit 4 serving as an access point establishes a wireless network, and informs of its own network identifier (SSID). Subsequently, the communication unit 32 of the processor 3 serving as a station searches for the network identifier (SSID) that has been informed, and connects to a desired network (access point). Because a network established with multiple devices is assumed, a covered range is wide, and it goes through strict identification steps, considering interference issues. Therefore, it can take time for establishing a connection. However, as for data communication, data can be transmitted and received between an access points and a station in respective different timings. The communication unit 32 may adopt communication using 4G wireless communication, other than Wi-Fi communication. The communication unit 32 may, of course, use other communications, such as 3G wireless communication, 5G wireless communication, worldwide interoperability for microwave access (WiMAX) (registered trademark) communication, and infrared communication (infrared data association (IrDA) (registered trademark)).

The recording unit 33 records various kinds of programs that are executed by the processor 3, data being processed, endoscopic image data, and the like. The recording unit 33 is constituted of a flash memory, a synchronous dynamic random access memory (SDRAM), a memory card, a solid state drive (SSD), or the like. Furthermore, the recording unit 33 includes an authentication-information recording unit 331 that records a device address of a device authenticated for mutual wireless communication and connectability determination information, and a processor-IP-address recording unit 332 that records a processor IP address identifying the processor 3.

The replaced-device-information recording unit 34 wirelessly transmits time of replacement of respective devices constituting the processor 3 to devices that are positioned within a predetermined range. The replaced-device-information recording unit 34 is constituted of a wireless tag, for example, a radio frequency identifier (RFID).

The position-information acquiring unit 35 acquires position information that is issued by the terminal device 5. The position-information acquiring unit 35 is constituted of, for example, an RFID reader or a communication module supporting Bluetooth communication.

The processor control unit 36 controls the respective devices constituting the processor 3 and the respective devices constituting the endoscope system 1. The processor control unit 36 is constituted of a central processing unit (CPU), and the like. The processor control unit 36 includes a connection determining unit 361, a display control unit 362, a communication control unit 363, a setting unit 364, and a drive control unit 365.

The connection determining unit 361 determines whether the terminal device 5 is a destination enable to perform mutual wireless communication based on a terminal IP address transmitted from the terminal device 5 and an authentication result.

The display control unit 362 controls a display mode of the display device 6. Specifically, the display control unit 362 causes the display device 6 to display an endoscopic image corresponding endoscopic image data subjected to the image processing by the video processing unit 31. Moreover, the display control unit 362 causes the display device 6 to display information indicating that the processor 3 and the terminal device 5 are enabled to perform mutual wireless communication when mutual wireless communication is established between the processor 3 and the terminal device 5.

The communication control unit 363 enables communication between the terminal device 5 and the respective peripheral devices based on the determination result of the connection determining unit 361 and the authentication result transmitted from the terminal device 5.

The setting unit 364 sets peripheral devices controllable by the terminal device 5 through the system controller 7 based on a level assigned to a registered user in the authentication result transmitted from the terminal device 5.

The drive control unit 365 controls drive of the peripheral device by controlling the system controller 7 based on a request signal or an operation signal input from the terminal device 5 through the communication unit 32.

Next, a configuration of the wireless unit 4 will be explained. The wireless unit 4 is connected to the server 200 through the network N100, and is connected to the processor and the terminal device 5 in accordance with a predetermined communication standard such that mutual communication is possible. The wireless unit 4 adopts Wi-Fi communication. Moreover, the wireless unit 4 is arranged around the processor 3, on a wall of the operating room 100, or the like.

Next, a configuration of the terminal device 5 will be explained. The terminal device 5 mutually communicates with the processor 3 in accordance with a predetermined communication standard, and receives endoscopic image data generated by the endoscope 2 and case image data from the server 200 through the wireless unit 4, to display them. Moreover, the terminal device 5 acquires at least one of a software program of each device constituting the endoscope system 1 and setting information of each device constituting the endoscope system 1 set by a registered user that can use the terminal device 5, from the processor 3 or the server 200 connected to the network N100. Furthermore, the terminal device 5 receives an input of an operation signal or a request signal to manipulate operations of the respective devices constituting the endoscope system 1 through the processor 3 or the wireless unit 4. A detailed configuration of the terminal device 5 will be described later.

Next, a configuration of the display device 6 will be explained. The display device 6 displays an image corresponding to image data input from the video processing unit 31 and various kinds of information of the endoscope system 1 under control of the display control unit 362. The display device 6 is constituted of a liquid crystal or an organic electroluminescence (EL) display monitor, a speaker that outputs sound externally, and the like.

The system controller 7 is wiredly or wirelessly connected to the processor 3, and independently controls each of the sound input unit 8, the ultrasound device 9, the insufflation device 10, the electrosurgical knife device 11, the printer 12, the room light 13, the electric operating table 14, and the wireless feeder device 15 according to an instruction input from the processor 3. Hereinafter, when either one of the sound input unit 8, the ultrasound device 9, the insufflation device 10, the electrosurgical knife device 11, the printer 12, the room light 13, the electric operating table 14, and the wireless feeder device 15 is referred to, it is simply denoted as “peripheral device”. Moreover, the system controller 7 is wiredly or wirelessly connected the respective peripheral devices. The system controller 7 is constituted of a CPU, a flash memory, or the like.

The sound input unit 8 collects sound output from a sound source or a speaker and converts into an analog sound signal (electrical signal), and subjects this sound signal to A/D conversion processing and gain adjustment processing, to generate digital sound data, and outputs the data to the processor 3 through the system controller 7, under control of the system controller 7. The sound input unit 8 is constituted of at least either one of microphone out of a unidirectional microphone, a nondirectional microphone, and a bidirectional microphone, an A/D convertor circuit, a signal processing circuit, and the like.

The ultrasound device 9 is connected to the endoscope 2, and transmits and receives ultrasonic waves through an ultrasound transducer provided at a distal end of the endoscope 2, under control of the system controller 7. Moreover, the ultrasound device 9 outputs ultrasound image data based on ultrasonic waves received through the endoscope 2 to the system controller 7. The ultrasound device 9 may generate ultrasound image data of a subject through a dedicated ultrasound probe.

The insufflation device 10 sends insufflation gas, for example, carbon dioxide, to an inside of a subject under control of the system controller 7.

The electrosurgical knife device 11 drives an electrosurgical knife by applying a predetermined voltage to the electrosurgical knife under control of the system controller 7.

The printer 12 outputs an image corresponding to image data input from the processor 3 under control of the system controller 7.

The room light 13 are arranged in the operating room 100 in plurality, and light up a subject and the operating room 100 at a predetermined illuminance, under control of the system controller 7. The room light 13 is constituted of a light emitting diode (LED) lamp, a dimmer switch, and the like.

As for the electric operating table 14, a subject is placed on an operating table. The electric operating table 14 changes a position and a posture of the subject by moving the operating table in a vertical direction and a horizontal direction, under control of the system controller 7. The electric operating table 14 is constituted of an operating table that is movable in a vertical direction and a horizontal direction, a motor that drives the operating table, and the like.

The wireless feeder device 15 supplies power to the terminal device 5 under control of the system controller 7. The wireless feeder device 15 is configured using at least one of an inductive coupling type, a magnetic field resonance type, an electric field coupling type, and a beam transmission/reception type.

The server 200 is arranged in the hospital but outside the operating room 100, and records endoscopic image data transmitted from the processor 3 or the terminal device 5 through the network N100, and patient ID identifying a patient in an associated manner. Moreover, when an image request signal requesting for case image data and endoscopic image data is received through the network N100 or the wireless unit 4, the server 200 transmits case image data and endoscopic image data to the processor 3 or the terminal device 5 that has issued the image request signal. The endoscopic image data includes moving image data and still image data (captured image data).

Configuration of Terminal Device

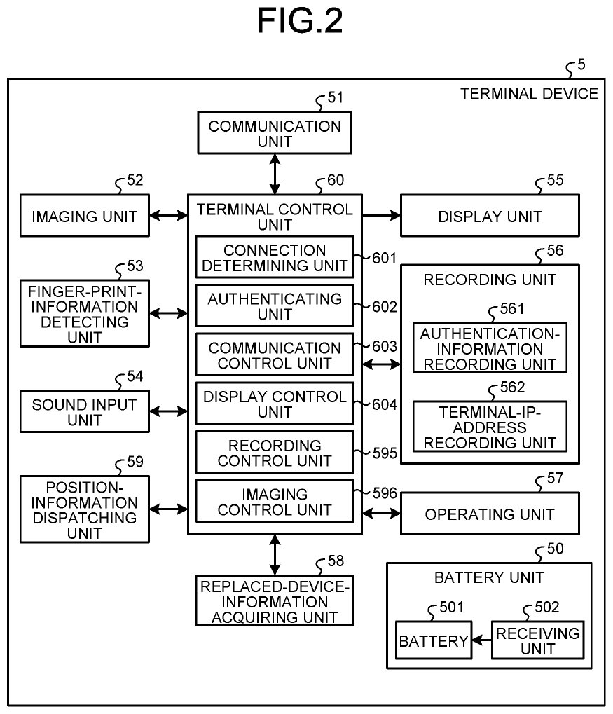

Next, a detailed configuration of the terminal device 5 explained in FIG. 1 will be explained. FIG. 2 is a block diagram illustrating a functional configuration of the terminal device 5.

The terminal device 5 illustrated in FIG. 2 includes a battery unit 50, a communication unit 51, an imaging unit 52, a finger-print-information detecting unit 53, a sound input unit 54, a display unit 55, a recording unit 56, an operating unit 57, a replaced-device-information acquiring unit 58, a position-information dispatching unit 59, and a terminal control unit 60.

The battery unit 50 includes a battery 501 that supplies power to respective parts constituting the terminal device 5, and a receiving unit 502 that receives electromagnetic waves fed by the wireless feeder device 15 to convert into an electric current to supply to the battery 501.

The communication unit 51 is constituted of a communication module, and performs mutual communication with the processor 3 in accordance with a predetermined communication standard. Moreover, the communication unit 51 performs mutual communication with the server 200 through the wireless unit 4 and the network N100 in the hospital. Wi-Fi communication is assumed to be used as the predetermined communication standard. The communication unit 51 may adopt communication using 4G wireless communication, other than the Wi-Fi communication. The communication unit 51 may, of course, use other communications, such as Bluetooth communication, BLE communication, 3G wireless communication, 5G wireless communication, WiMAX communication, and infrared communication.

The imaging unit 52 images a user of the terminal device 5 to generate image data, and outputs this image data to the terminal control unit 60 under control of the terminal control unit 60. The imaging unit 52 is implemented by using an image sensor of a CCD, a CMOS, and the like, an image processing engine implemented by using A/D conversion processing, an FPGA, a GPU, and the like. By arranging an infrared lamp that can irradiate infrared light, and an image sensor having pixels capable of imaging infrared light irradiated by this infrared lamp in the imaging unit 52, it may be configure to acquire projections and depressions of a facial surface of a user.

The finger-print-information detecting unit 53 detects finger print information of a finger of a user externally touched, and outputs this detection result to the terminal control unit 60. The finger-print-information detecting unit 53 is constituted of a finger print sensor. The finger-print-information detecting unit 53 may be, for example, of a sliding type, other than a press type. The finger-print-information detecting unit 53 may, of course, detect vein patterns of a user, other than finger prints.

The sound input unit 54 collects a sound output from a sound source or a speaker to convert into an analog sound signal (electrical signal), and subjects this sound signal to A/D conversion processing or gain adjustment processing, to generate digital sound data, and outputs the data to the terminal control unit 60, under control of the terminal control unit 60. The sound input unit 54 is constituted of either one of microphone out of a unidirectional microphone, a nondirectional microphone, and a bidirectional microphone, an A/D converter circuit, a signal processing circuit, and the like.

The display unit 55 displays image data and various kinds of data input from the terminal control unit 60. The display unit 55 is constituted of a display panel of a liquid crystal, an organic EL, or the like.

The recording unit 56 records various kinds of programs executed by the terminal device 5, data being processed, image data, and the like. The recording unit 56 is constituted of a flash memory, an SSD, a memory card, and the like. Moreover, the recording unit 56 includes an authentication-information recording unit 561, a terminal-IP-address recording unit 562 that records a terminal IP address identifying the terminal device 5.

The operating unit 57 receives an input of an instruction signal according to an operation by a user. The operating unit 57 is constituted of a touch panel, a button, a switch, and the like.

The replaced-device-information acquiring unit 58 acquires radio waves transmitted from the replaced-device-information recording unit 34 provided in the processor 3, to output to the terminal control unit 60. The replaced-device-information acquiring unit 58 is constituted of an RFID reader.

The position-information dispatching unit 59 transmits position information regarding a position of the terminal device 5 to a predetermined distance. Specifically, the position-information dispatching unit 59 transmits the position information to a reachable distance in the operating room 100. The position-information dispatching unit 59 is constituted of, for example, an RFID or a communication module supporting the Bluetooth communication.

The terminal control unit 60 overall controls the respective parts constituting the terminal device 5. Moreover, the terminal control unit 60 analyzes sound data input from the sound input unit 54, and generates a sound command based on this analysis result, to transmit to the processor 3. The terminal control unit 60 is constituted of a CPU, or the like. The terminal control unit 60 includes a connection determining unit 601, an authenticating unit 602, a communication control unit 603, a display control unit 604, a recording control unit 605, and an imaging control unit 606.

The connection determining unit 601 determines whether the processor 3 is a destination enabled to perform mutual wireless communication, based on authentication information received by the communication unit 51 from the processor 3.

The authenticating unit 602 authenticates whether a user of the terminal device 5 is a registered user that has been pre-registered. Specifically, the authenticating unit 602 performs authentication by acquiring at least either one of a face image of a user of the terminal device 5, biometric information of the user, and gesture information of the user. For example, the authenticating unit 602 determines whether features of a face image of a user appearing in an image corresponding to image data generated by the imaging unit 52 coincide with features of the face of the registered user recorded in the recording unit 56.

The communication control unit 603 enables mutual wireless communication between the processor 3 and the terminal device 5, or mutual wireless communication between the network N100 and the terminal device 5 based on a determination result of the connection determining unit 601 and an authentication result of the authenticating unit 602.

The display control unit 604 controls a display mode of the display unit 55. Specifically, the display control unit 604 causes the display unit 55 to display an endoscopic image corresponding to endoscopic image data and a case image corresponding to case image data, in such a manner that enables comparison thereof.

The recording control unit 605 causes the recording unit 56 to record a doctor's round time for a patient included in schedule information acquired by the communication control unit 603 and endoscopic image data, in an associated manner.

The imaging control unit 606 enables an imaging function of the imaging unit 52 for the user of the terminal device 5 when the user is authenticated as a registered user by the authenticating unit 602.

Processing of Processor

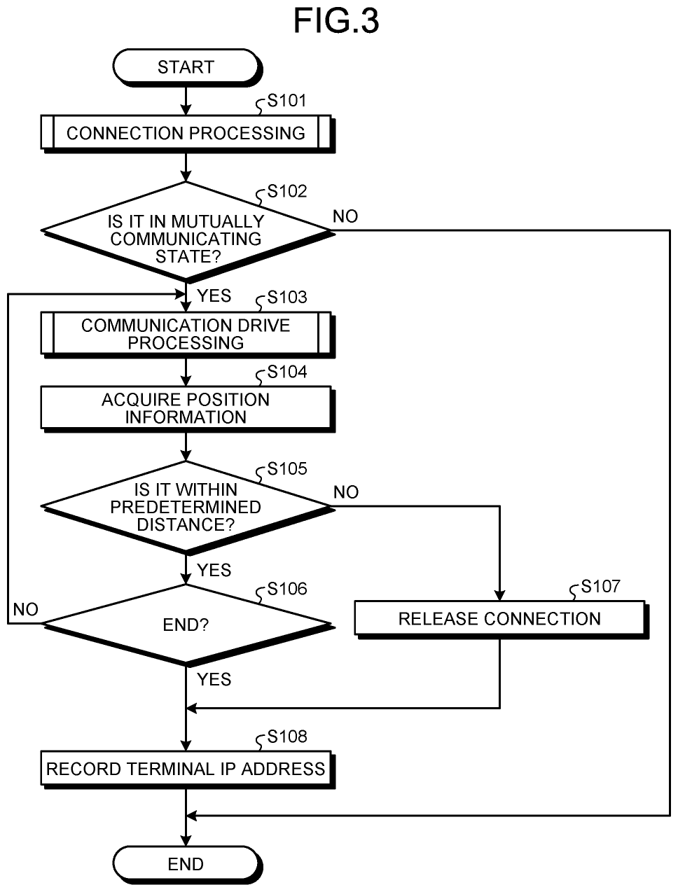

Next, processing performed by the processor 3 will be explained. FIG. 3 is a flowchart showing an overview of the processing performed by the processor 3.

As shown in FIG. 3, the processor 3 performs connection processing to establish a connection to perform mutual communication with the terminal device 5 (step S101). After step S101, the processor 3 proceeds to step S102 described later.

Connection Processing

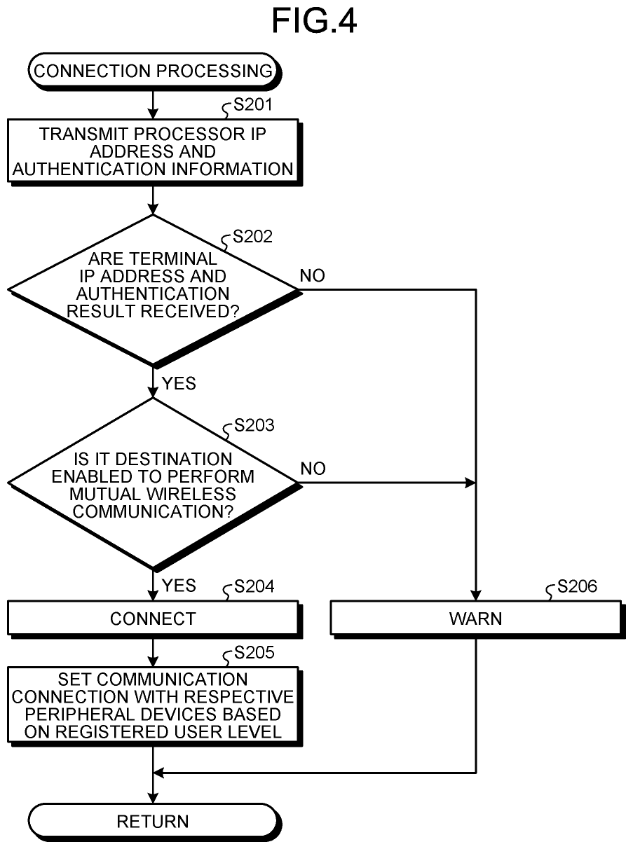

Next, details of the connection processing explained in step S101 in FIG. 3 will be explained. FIG. 4 is a flowchart showing details of the connection processing.

As shown in FIG. 4, first, the communication control unit 363 transmits a processor IP address (SSID) and authentication information to the terminal device 5 through the communication unit 32 (step S201). The authentication information herein is information to request for an authentication result of the authenticating unit 602 that serves as a password to perform mutual wireless communication of the terminal device 5.

Subsequently, when the terminal IP address and the authentication result authenticated by the authenticating unit 602 are received from the terminal device 5 through the communication unit 32 (step S202: YES), the processor 3 proceeds to step S203 described later. On the other hand, when the terminal IP address and the authentication result authenticated by the authenticating unit 602 are received from the terminal device 5 through the communication unit 32 (step S202: NO), the processor 3 returns to the main routine in FIG. 3.

At step S203, the connection determining unit 361 determines whether the terminal device 5 is a destination enabled to perform mutual wireless communication based on the terminal IP address and the authentication result authenticated by the authenticating unit 602 (step S203). Specifically, the connection determining unit 361 determines whether the terminal IP address and the authentication result authenticated by the authenticating unit 602 received by the communication unit 32 coincide with authentication information recorded in the authentication-information recording unit 331, and determines that the terminal device 5 is a destination enabled to perform mutual wireless communication when they coincide with each other, on the other hand, determines that the terminal device 5 is not a destination enabled to perform mutual wireless communication when they do not coincide with each other. When the connection determining unit 361 determines that the terminal device 5 is a destination enabled to perform mutual wireless communication (step S203: YES), the processor 3 proceeds to step S204 described later. On the other hand, when the connection determining unit 361 determines that the terminal device 5 is not a destination enabled to perform mutual wireless communication (step S203: NO), the processor 3 proceeds to step S206 described later.

At step S204, the communication control unit 363 connects the processor 3 and the terminal device 5 such that mutual communication is possible. Thus, the processor 3 becomes possible to mutually communicate with the terminal device 5. In this case, the display control unit 362 may cause the display device 6 to display that mutual wireless communication between the processor 3 and the terminal device 5 are enabled. That is, the display control unit 362 functions as an informing unit.

Subsequently, the setting unit 364 sets a communication connection with each of plural peripheral devices that can be operated by the terminal device 5 through the system controller 7, based on a level assigned to the registered user of the authentication result received from the terminal device 5 (step S205). Specifically, the setting unit 364 sets all peripheral devices to the peripheral device that can be operated through the terminal device 5 when the level of the registered user is a doctor level, and sets, on the other hand, only a designated peripheral device to the peripheral device that can be operated through the terminal device 5 when the level of the registered user is a nurse level. For example, a peripheral device that is not related to an operation, more specifically, the printer 12, the room light 13, the wireless feeder device 15, and the like are set to be operable. Of course, the setting unit 364 may be configured to be able to set the peripheral device that can be operated by the terminal device 5 more precisely based on a level of the registered user. After step S205, the processor 3 returns to the main routine in FIG. 3.

At step S206, the display control unit 362 causes the display device 6 to display a warning indicating that the terminal device 5 is not a destination enabled to perform mutual wireless communication. The display control unit 362 controls the display device 6 to display the warning indicating that the terminal device 5 is not a destination enabled to perform mutual wireless communication, but the warning indicating that the terminal device 5 is not a destination enabled to perform mutual wireless communication may be informed by, for example, a not illustrated speaker or the like. That is, the display control unit 362 functions as an informing unit that informs that mutual wireless communication is not possible between the processor 3 and the terminal device 5. After step S206, the processor 3 returns to the main routine in FIG. 3.

Returning back to FIG. 3, explanation of step S102 and later will be continued.

At step S102, when the processor 3 and the terminal device 5 are enabled to perform mutual communication (step S102: YES), the processor 3 performs communication driving processing to drive a peripheral device according to an operation for which an input is received by the terminal device 5 (step S103). After step S103, the processor 3 proceeds to step S104 described later. On the other hand, when the processor 3 and the terminal device 5 are not enabled to perform mutual communication (step S102: NO), the processor 3 ends the processing.

Communication Driving Processing

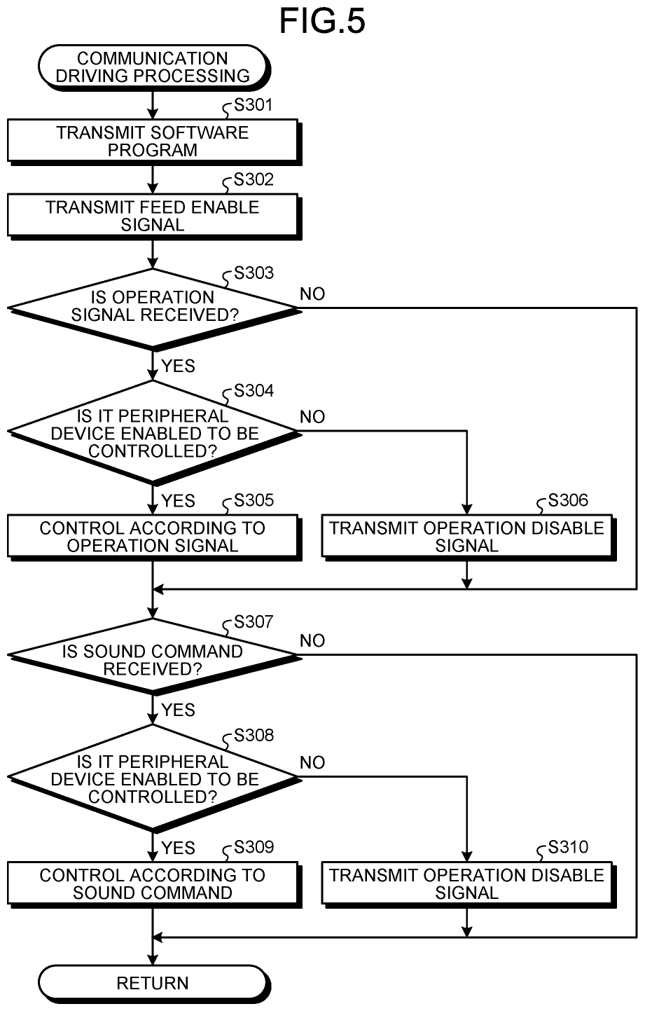

Next, details of the communication driving processing explained at step S103 in FIG. 3 will be explained. FIG. 5 is a flowchart for explaining details of the communication driving processing.

As shown in FIG. 5, the communication control unit 363 causes the communication unit 32 to transmit a software program of the peripheral device and the processor 3 to the terminal device 5 (step S301). The software program includes setting information including setting parameters of initial values in a peripheral device and setting parameters that are set to a peripheral device at a previous operation by a user, other than program updates of the peripheral device and the processor 3.

Subsequently, the drive control unit 365 transmits a feed enable signal to the wireless feeder device 15 through the system controller 7 (step S302). Thus, an electric power can be supplied to the terminal device 5 positioned in the operating room 100.

Thereafter, when the communication unit 32 receives an operation signal operating the peripheral device from the terminal device 5 (step S303: YES), the processor 3 proceeds to step S304 described later. On the other hand, when the communication unit 32 does not receive an operation signal operating the peripheral device from the terminal device 5 (step S303: NO), the processor 3 proceeds to step S307.

At step S304, when the operation signal received from the terminal device 5 is for a peripheral device that is enabled to be controlled by settings of the setting unit 364 (step S304: YES), the drive control unit 365 performs a control according to the operation signal with respect to the peripheral device according to the operation signal (step S305). For example, the drive control unit 365 changes the illuminance of the room light 13 through the system controller 7 when the operation signal is an operation signal to change the illuminance of the room light 13. After step S305, the processor 3 proceeds to step S307 described later.

At step S304, the operation signal received from the terminal device 5 is not for a peripheral device that is enabled to be controlled by the settings by the setting unit 364 (step S304: NO), the communication control unit 363 causes the communication unit 32 to transmit an operation disable signal indicating that the peripheral device according to the operation signal is not operable, to the terminal device 5 (step S306). After step S306, the processor 3 proceeds to step S307 described later.

At step S307, when the communication unit receives a sound command operating a peripheral device from the terminal device 5 (step S307: YES), the processor 3 proceeds to step S308 described later. On the other hand, when the communication unit 32 does not receive a sound command operating a peripheral device from the terminal device 5 (step S307: NO), the processor 3 returns to the main routine in FIG. 3.

At step S308, when the sound command received from the terminal device 5 is for a peripheral device that is enabled to be controlled by the settings of the setting unit 364 (step S308: YES), the drive control unit 365 performs a control according to the sound command with respect to the peripheral device according to the sound command (step S309). For example, the drive control unit 365 changes the illuminance of the room light 13 through the system controller when the sound command is an instruction to change the illuminance of the room light 13. After step S309, the processor 3 returns to the main routine in FIG. 3.

At step S308, when the sound command received from the terminal device 5 is not for a peripheral device that is allowed to be operated by settings of the setting unit 364 (step S308: NO), the communication control unit 363 causes the communication unit 32 to transmit the operation disable signal indicating that the peripheral device according to the sound command is not operable, to the terminal device 5 (step S310). After step S310, the processor 3 returns to the main routine in FIG. 3.

Returning back to FIG. 3, explanation of step S104 and later will be continued.

At step S104, the position-information acquiring unit 35 acquires position information of the terminal device 5. Specifically, the position-information acquiring unit 35 acquires position information of the terminal device 5 by receiving radio waves emitted from the position-information dispatching unit 59 of the terminal device 5.

Subsequently, the connection determining unit 361 determines whether the terminal device 5 is positioned within a predetermined distance from the processor 3 based on the position information of the terminal device 5 acquired by the position-information acquiring unit 35 (step S105). Specifically, the connection determining unit 361 determines whether the position-information acquiring unit 35 has acquired the position information of the terminal device 5. When the connection determining unit 361 determines that the terminal device 5 is positioned within the predetermined distance from the processor 3 (step S105: YES), the processor 3 proceeds to step S106 described later. On the other hand, when the connection determining unit 361 determines that the terminal device 5 is not positioned within the predetermined distance from the processor 3 (step S105: NO), the processor 3 proceeds to step S107 described later.

At step S106, when an instruction signal instructing an end is input from the terminal device 5 (step S106: YES), the processor 3 proceeds to step S108 described later. On the other hand, when the instruction signal indicating an end is not input from the terminal device 5 (step S106: NO), the processor 3 returns to step S103 described above.

At step S107, the communication control unit 363 releases connection between the processor 3 and the terminal device 5 in a communicating state. Thus, even when the terminal device 5 is moved outside the operating room 100, it is possible to prevent the peripheral devices from being operated by remote operation. After step S107, the processor proceeds to step S108 described later.

At step S108, the communication control unit 363 records a terminal IP address of the terminal device 5 in the recording unit 33. Thus, it is possible to connect the processor 3 and the terminal device 5 into a communicating state swiftly when the power of the terminal device 5 is turned on in the operating room 100. After step S108, the processor 3 ends the processing.

According to one embodiment described above, the communication control unit 363 enables mutual wireless communication between the processor 3 and the terminal device 5 based on a determination result of the connection determining unit 361 and an authentication result of the authenticating unit 602 transmitted from the terminal device 5 and, therefore, both efficiency and information security can be considered.

Moreover, according to one embodiment, when communication between the terminal device 5 and multiple peripheral devices are enabled, the communication control unit 363 can cause the communication unit 32 to transmit a software program to the terminal device 5 and, therefore, the peripheral devices can be operated promptly by using the terminal device 5, without setting parameters of the peripheral devices each time.

Furthermore, according to one embodiment, the setting unit 364 sets multiple peripheral devices that can be operated by the terminal device 5 through the system controller 7 based on a level assigned to a registered user in an authentication result of the authenticating unit 602 that is transmitted from the terminal device 5 and, therefore, security according to a user can be set.

Moreover, according to one embodiment, when the communication control unit 363 enables communication between the terminal device 5 and multiple peripheral devices, the drive control unit 365 causes the wireless feeder device 15 to supply power to the terminal device 5 through the system controller 7 and, therefore, it is possible to prevent power from being supplied to the terminal device 5 for which security is not guaranteed.

Furthermore, according to one embodiment, when the communication control unit 363 enables communication between the terminal device 5 and multiple peripheral devices, in a case in which the communication unit 32 receives an operation signal operating any one of multiple peripheral devices from the terminal device 5, the drive control unit 365 drives a peripheral device according to the operation signal through the system controller 7 and, therefore, security is guaranteed.

Moreover, according to one embodiment, when the communication control unit 363 enables communication between the terminal device 5 and multiple peripheral devices, in a case in which the communication unit 32 receives an operation signal operating any one of multiple peripheral devices from the terminal device 5, the drive control unit 365 drives a peripheral device according to a sound command through the system controller 7 and, therefore, security is guaranteed.

Furthermore, according to one embodiment, when it becomes impossible to acquire position information of the terminal device 5 of the position-information acquiring unit 35, the communication control unit 363 releases connection between the terminal device 5 and multiple peripheral devices and, therefore, even when the terminal device 5 is moved outside the operating room 100, it is possible to prevent it from being remotely operated from outside, and security is guaranteed.

Moreover, according to one embodiment, when the wireless unit 4 receives a terminal IP address information from the terminal device 5, connectability determination information indicating whether mutual wireless communication with the terminal device 5 is possible may be transmitted to the terminal device 5. Thus, operation of the endoscope system 1 by the terminal device 5 through the wireless unit 4 is enabled.

Furthermore, according to one embodiment, the wireless unit 4 may hold the connectability determination information, and may perform an automatic connection between the terminal device 5 and the wireless unit 4 based on the connectability determination information after power of the terminal device 5 is turned on. Thus, operability can be improved. Of course, the terminal device 5 may also perform an automatic connection between the terminal device 5 and the wireless unit 4 based on the connectability determination information after power of the terminal device 5 is turned on.

Moreover, according to one embodiment, mutual wireless communication between the server 200 connected to the network N100 and the terminal device 5 may be enabled. Thus, both efficiency and information security can be considered.

### Other Embodiments

Plural components disclosed in one embodiment described above can be combined appropriately. For example, some components may be omitted from all of the components described in one embodiment described above. Furthermore, the components explained in one embodiment described above may be combined.

Furthermore, although a light source device is provided in a processor in one embodiment, it may be formed separately.

Moreover, although one embodiment is for an endoscope system, it is also applicable to, for example, a capsule endoscope, a video microscope for imaging a subject, a mobile phone having an imaging function, and a tablet terminal having an imaging function.

Furthermore, although one embodiment is for an endoscope system including a medical endoscope, it is also applicable to an endoscope system including an industrial endoscope.

Moreover, “unit” used in description above can be read as “means”, “circuit”, or the like. For example, the control unit can be read as control means or control circuit.

Furthermore, a program to be executed in one embodiment is recorded on a computer-readable recording medium, such as a compact disk read-only memory (CD-ROM, a flexible disk (FD), a compact disk recordable (CD-R), a digital versatile disk (DVD), a universal serial bus (USB) medium, and a flash memory, in a file data of a installable format or an executable format, to be provided.

Moreover, a program to be executed by the endoscope system of on embodiment may be stored in a computer connected to a network, such as the Internet, and be provided by being downloaded through the network. Furthermore, a computer program to be executed by the endoscope system of one embodiment may be provided or distributed through a network, such as the Internet.

Although a sequential relation of processing among steps is specified by using expressions, such as “first”, “thereafter”, and “subsequently”, in the explanation of the flowcharts in the present specification, it is noted that the order of processing is not uniquely specified by those expressions. That is, the order of processing in the flowcharts described in the present specification may be changed within a range not causing a contradiction. Furthermore, the computer program is not limited to be of simple branch processing as described above. Branching may be performed by generally determining more determination points. In that case, a technique of artificial intelligence that achieves machine learning while prompting a user for manual operations to repeat training may be used in combination. Moreover, it may be configured to learn operating patterns performed by many specialists, and to perform deep learning by applying further complicated conditions.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope system comprising:
a processor configured to perform image processing on endoscopic image data acquired by an endoscope that observes inside a subject, the processor being connectable to a peripheral device; and
a terminal device configured to communicate with the processor, wherein
the terminal device is configured to transmit terminal identification information identifying the terminal device, and an authentication result indicating whether a user of the terminal device is a registered user that has been pre-registered,
the processor includes
a communication circuit configured to
transmit processor identification information identifying the processor, and authentication information that enables mutual communication, to the terminal device, and
receive the terminal identification information and the authentication result from the terminal device;
a connection determining circuit configured to determine whether the terminal device is a destination that is enabled to perform mutual communication based on the authentication result received by the communication circuit; and
a communication control circuit configured to enable communication between the terminal device and the peripheral device based on a determination result of the connection determining circuit and on the authentication result.

2. The endoscope system according to claim 1, wherein
the processor is connected to a network to which a server storing a software program relating to settings of the peripheral device is connected, and
the communication control circuit is further configured to cause the terminal device to transmit the software program to the communication circuit when the communication control circuit has enabled the communication between the terminal device and the peripheral device.

3. The endoscope system according to claim 1, further comprising
a system controller that is connected to the processor and the peripheral device, the system controller being configured to control drive of the peripheral device, wherein
the processor further includes a setting circuit configured to set the peripheral device that is enabled to be operated by the terminal device through the system controller based on a level assigned to the registered user of the authentication result.

4. The endoscope system according to claim 3, wherein
the processor further includes a drive control circuit configured to control drive of the peripheral device through the system controller,
the peripheral device is a wireless feeder device configured to supply power wirelessly to the terminal device,
the drive control circuit is configured to cause the wireless feeder device to supply power to the terminal device through the system controller when the communication control circuit has enabled the communication between the terminal device and the peripheral device.

5. The endoscope system according to claim 3, wherein
the processor further includes a drive control circuit configured to control drive of the peripheral device through the system controller,
when the communication control circuit has enabled the communication between the terminal device and the peripheral device and when the communication circuit has received an operation signal operating the peripheral device from the terminal device, the drive control circuit is configured to drive the peripheral device according to the operation signal through the system controller.

6. The endoscope system according to claim 3, wherein
the terminal device is configured to receive an input of a sound command operating the peripheral device by sound input,
the processor further includes a drive control circuit configured to control drive of the peripheral device through the system controller, and
when the communication control circuit has enabled the communication between the terminal device and the peripheral device and when the communication circuit has receive the sound command from the terminal device, the drive control circuit is configured to drive the peripheral device according to the sound command through the system controller.

7. The endoscope system according to claim 1, wherein
the terminal device is configured to dispatch position information relating to a position of the terminal device to a predetermined distance,
the processor further includes a position-information acquiring circuit configured to acquire the position information issued by the terminal device, and
the communication control circuit is configured to release connection between the terminal device and the peripheral device when the position-information acquiring circuit becomes impossible to acquire the position information.

8. The endoscopes system according to claim 1, wherein
the terminal device is configured to acquire any one or more of a face image of the user, biometric information of the user, and gesture information of the user, to perform authentication.

9. The endoscope system according to claim 1, wherein
the mutual communication is mutual wireless communication.

10. The endoscope system according to claim 9, further comprising
a wireless unit that is connected to a network, the wireless unit being enabled to perform mutual wireless communication with the terminal device and the processor, wherein
when identification information is received from the terminal device, the wireless unit is configured to transmit connectability determination information indicating whether mutual wireless communication is possible with the terminal device, to the processor.

11. The endoscope system according to claim 10, wherein
the terminal device or the wireless unit is configured to hold the connectability determination information, and perform an automatic connection between the terminal device and the wireless unit based on the connectability determination information after power is turned on.

12. The endoscope system according to claim 1, wherein
the processor further includes an informing circuit configured to inform that mutual wireless communication between the processor and the terminal device is enabled by the communication control circuit.

13. The endoscope system according to claim 1, wherein
the processor is connectable to a plurality of peripheral devices.

14. A processor that performs image processing on endoscopic image data acquired by an endoscope that observes inside a subject, the processor being connectable to a peripheral device, the processor comprising:
a communication circuit configured to
transmit processor identification information identifying the processor, and authentication information that enables mutual communication, to the terminal device configured to communicate with the processor, and
receive terminal identification information identifying the terminal, and an authentication result indicating whether a user of the terminal device is a registered user that has been pre-registered, from the terminal device;
a connection determining circuit configured to determine whether the terminal device is a destination enabled to perform mutual communication based on the authentication result received by the communication circuit; and
a communication control circuit configured to enables communication between the terminal device and the peripheral device based on a determination result of the connection determining circuit and on the authentication result.

15. A control method that is performed by an endoscope system including a processor that performs image processing on endoscopic image data acquired by an endoscope that observes inside a subject, the processor being connectable to a peripheral device, and a terminal device configured to communicate with the processor, the method comprising:
transmitting processor identification information identifying the processor, and authentication information that enables mutual communication, to the terminal device;
receiving terminal identification information identifying the terminal device, and an authentication result indicating whether a user of the terminal device is a registered user that has been pre-registered, from the terminal device;
determining whether the terminal device is a destination that is enabled to perform mutual communication based on the authentication result; and
enabling communication between the terminal device and the peripheral device based on the determination result and on the authentication result.

16. A non-transitory computer-readable recording medium with an executable program stored thereon, the program instructing an endoscope system including a processor that performs image processing on endoscopic image data acquired by an endoscope that observes inside a subject, the processor being connectable to a peripheral device, and a terminal device configured to communicate with the processor to perform:
transmitting processor identification information identifying the processor, and authentication information that enables mutual communication, to the terminal device;
receiving terminal identification information identifying the terminal device, and an authentication result indicating whether a user of the terminal device is a registered user that has been pre-registered, from the terminal device;
determining whether the terminal device is a destination that is enabled to perform mutual communication based on the authentication result; and
enabling communication between the terminal device and the peripheral device based on the determination result and on the authentication result.
